(19) United States
(12) Patent Application Publication
(10) Pub. No.: US 2026/0256454 A1
(43) Pub. Date: Sep. 3, 2026

(54) SYSTEMS AND METHODS FOR IMAGING SCREENING

(71) Applicant: KONINKLIJKE PHILIPS N.V., EINDHOVEN (NL)

(72) Inventors: Muhammad Usman Ghani, Boston, MA (US); Hyeon Woo Lee, Cambridge, MA (US); Jonathan Fincke, Belmont, MA (US); Balasundar Iyyavu Raju, North Andover, MA (US)

(21) Appl. No.: 18/993,293

(22) PCT Filed: Jul. 10, 2023
(86) PCT No.: PCT/EP2023/069078

§ 371 Date: Jan. 10, 2025

Related U.S. Application Data
(60) Provisional application No. 63/359,931, filed on Jul. 11, 2022.

(51) Int. Cl. A61B 8/00

(52) U.S. Cl. CPC A61B 8/5223; A61B 8/466; A61B 8/5292

(57) ABSTRACT

An ultrasound imaging system may perform a scan/exam completeness score for each zone/region explored during an ultrasound exam. In some examples, a list of tasks for the zone being examined may be provided on a user interface. The system may automatically detect the tasks completion based on anatomical features detected in the images. The system may provide a scan score/meter. In some examples, the anatomical features, scan completeness, scan quality, and/or other metrics may be determined, at least in part, by one or more machine learning models.

### TECHNICAL FIELD

The present disclosure pertains to imaging systems and methods for monitoring the progress of an imaging exam, more specifically, the present disclosure pertains to monitoring the progress of an ultrasound exam.

### BACKGROUND

Ultrasound exams are valuable for a wide variety of diagnostic purposes such as fetal development monitoring, cardiac valve health assessment, liver disease monitoring, and detecting internal bleeding. Accurate diagnosis from ultrasound images rely on capturing correct views of anatomy as well as quality of the images (e.g., resolution). The diagnostic value of the images may decrease if insufficient and/or incorrect views of anatomy are obtained or the images are poor quality (e.g., blurred due to motion artefacts). Accordingly, techniques for ensuring completeness of ultrasound exams and quality of ultrasound images may be desirable.

### SUMMARY

The present disclosure addresses the challenges of conducting FAST exams by determining a scan completeness score for each zone/region explored during the FAST exam. The system described herein may provide a list of tasks for the zone being examined. The system may automatically detect task completion based on anatomical features detected in the imagery and may provide a scan score/meter as feedback to the user. This may enhance exam quality and improve sensitivity of FAST exam irrespective of experience level. In some applications, the system may be used as a tool for physician training and/or used for automated skill level analysis of physicians during and/or after the training.

According to at least one example of the present disclosure, an ultrasound imaging system may include an ultrasound probe configured to acquire an ultrasound image from a subject, a display configured to provide the ultrasound image, and a processor configured to receive the ultrasound image, determine whether one or more anatomical features are included in the ultrasound image, based on the anatomical features included in the ultrasound image, determine a status of a task, and provide display data to the display based on the status of the task, wherein the display is further configured to provide a visual indication of the status based on the display data.

In some examples, the processor is further configured to determine a zone within the subject where the ultrasound image was acquired and provide second display data to the display based on a set of tasks associated with the zone, wherein the display is further configured to provide a second visual indication of the set of tasks based on the second display data. In some examples, a completed task of the set of tasks is displayed differently than an uncompleted task of the set of tasks.

In some examples, the processor implements a machine learning model configured to determine whether the anatomical features are included in the ultrasound image.

In some examples, the processor is further configured to determine whether the ultrasound image is high quality or low quality prior to determining whether the anatomical features are included.

In some examples, the ultrasound imaging system further includes a user interface configured to receive an input from the user, wherein the input indicates an exam type, a zone of the subject, or a combination thereof.

In some examples, the ultrasound imaging system further includes a memory, wherein the processor is further configured to cause the ultrasound imaging system to save to the memory the ultrasound image, a future acquired ultrasound image, or a combination thereof.

According to at least one example of the disclosure, A non-transitory computer readable medium encoded with instructions that when executed, may cause an ultrasound imaging system to determine whether one or more anatomical features are included in an ultrasound image, based on the anatomical features included in the ultrasound image, determine a status of a task, generate display data based on the status of the task, and cause a display of the ultrasound imaging system to provide a visual indication of the status based on the display data.

According to at least one example of the disclosure, a method may include acquiring ultrasound images from a subject with an ultrasound probe, determining with at least one processor, whether one or more anatomical features are included in the ultrasound images, based on the anatomical features included in the ultrasound images, determining a status of a task, and providing on a display a visual indication of the status.

In some examples, the method may further include determining a zone within the subject where the ultrasound images were acquired and providing a second visual indication of a set of tasks based on the zone. In some examples, a completed task of the set of tasks is displayed differently than an uncompleted task of the set of tasks. In some examples, the completed task is a different color than the uncompleted task.

In some examples, the method further includes determining whether the ultrasound images are high quality or low quality prior to determining whether the anatomical features are included.

In some examples, the method further includes determining whether the ultrasound images are high quality or low quality based, at least in part, on whether the anatomical features are included.

In some examples, the method further includes saving to a memory the ultrasound images, future acquired ultrasound images, or a combination thereof.

In some examples, the ultrasound images include a three-dimensional (3D) dataset.

In some examples, the method further includes determining, based on at least one ultrasound image, whether the task, a set of tasks, or a combination thereof can be completed by acquiring a current ultrasound image from a current location of the ultrasound probe. In some examples, the method further includes providing a prompt via a user interface to change a location of the ultrasound probe.

In some examples, the method further includes, computing a score indicating a degree of completeness of the task, a degree of completeness of an exam, or a combination thereof. In some examples, the score is based, at least in part, on a confidence score provided by a machine learning model. In some examples, the score indicating a degree of completeness of the exam is based, at least in part, on a number of tasks completed out of a total number of tasks.

### BRIEF DESCRIPTION OF THE DRAWINGS

FIG. 1 is a block diagram of an ultrasound system in accordance with examples of the present disclosure.

FIG. 2 is a block diagram illustrating an example processor in accordance with examples of the present disclosure.

FIG. 3 is a block diagram of a process for training and deployment of a neural network in accordance with examples of the present disclosure.

FIG. 4 shows example predictions made by a machine learning model compared to ground truth in accordance with examples of the present disclosure

FIG. 5 is a flowchart providing an overview of exam completeness analysis in accordance with the examples of the present disclosure.

FIG. 6 shows an example of a display providing a visual indication of tasks to be completed and completed in accordance with examples of the present disclosure.

FIG. 7 is a flow chart of a method according to examples of the present disclosure.

### DETAILED DESCRIPTION

The following description of certain embodiments is merely exemplary in nature and is in no way intended to limit the invention or its applications or uses. In the following detailed description of embodiments of the present systems and methods, reference is made to the accompanying drawings which form a part hereof, and which are shown by way of illustration specific embodiments in which the described systems and methods may be practiced. These embodiments are described in sufficient detail to enable those skilled in the art to practice presently disclosed systems and methods, and it is to be understood that other embodiments may be utilized and that structural and logical changes may be made without departing from the spirit and scope of the present system. Moreover, for the purpose of clarity, detailed descriptions of certain features will not be discussed when they would be apparent to those with skill in the art so as not to obscure the description of the present system. The following detailed description is therefore not to be taken in a limiting sense, and the scope of the present system is defined only by the appended claims.

As an example of an ultrasound exam, the Focused Assessment with Sonography for Trauma (FAST) exam is a rapid ultrasound exam conducted in trauma situations to assess patients for free-fluid. Different zones (e.g., region of the body) of a subject are scanned to search for free-fluid (e.g., blood) within the subject. Zones typically include the right upper quadrant (RUQ), the left upper quadrant (LUQ), and the pelvis (SP). Zones may further include the lung and heart. Each zone may include one or more regions of interest (ROIs), which may be organs or particular views of organs. For example, a typical FAST exam includes images of the kidney, liver, liver tip, diaphragm, kidney-liver interface, diaphragm-liver interface, and volume fanning acquired from the RUQ zone. In another example, during a typical FAST exam a subxiphoid view of the heart is acquired.

The FAST exam is a highly important step in triaging patient care in trauma situations. The FAST exam is a highly valuable diagnostic tool in the trauma situations. For example, detection of free-fluid may allow diagnosis of internal bleeding and/or trauma to internal organs. However, different studies have reported a large sensitivity range for the FAST exam. The major factor contributing to low sensitivity exams is insufficient scanning by physicians. Inexperience or less experienced physicians often do not scan enough to interrogate the entire abdominal volume, leaving the free fluid exploration task incomplete. Studies have found that novice users spend more time on FAST exam and imaged fewer points of interest as compared to experienced users. These studies reported that each point of care ultrasound (POCUS) exam typically require feedback from an expert on where the exam has been adequately completed. This requirement is a significant hurdle due to the small number of experience users. As a result, the overall workflow becomes slow and inefficient.

Complete ultrasound screening of a large volume and/or screening of multiple regions of interest (ROIs), such as during an ultrasound exam, such as FAST exam, may require acquisition of many ultrasound images from multiple view directions and scan windows. It may be difficult for a user (e.g., sonographer) to keep track of which images have been acquired, where within a subject images have been acquired, whether they have been acquired with sufficient quality to identify any potentially clinically significant issues, where gaps in imaging coverage exist, and/or what fraction of the volume or and/or ROIs have been scanned sufficiently. The present disclosure describes image data processing, visualization, feedback and guidance to address these problems. In some examples, a machine learning model may be trained and deployed to determine what ROIs have been imaged. The determinations may be used detect and/or score completion of tasks within a scan (e.g., imaging an ROI and/or a view of an ROI), classify and/or score completeness of the scan.

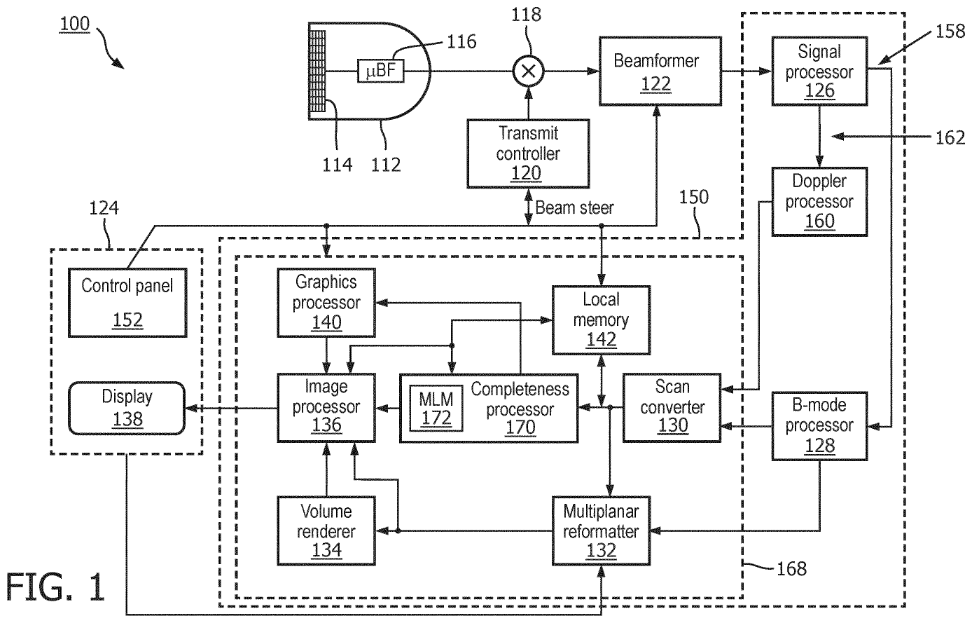

FIG. 1 shows a block diagram of an ultrasound imaging system 100 constructed in accordance with the examples of the present disclosure. An ultrasound imaging system 100 according to the present disclosure may include a transducer array 114, which may be included in an ultrasound probe 112, for example an external probe or an internal probe such as an intravascular ultrasound (IVUS) catheter probe. In other embodiments, the transducer array 114 may be in the form of a flexible array configured to be conformally applied to a surface of subject to be imaged (e.g., patient). The transducer array 114 is configured to transmit ultrasound signals (e.g., beams, waves) and receive echoes responsive to the ultrasound signals. A variety of transducer arrays may be used, e.g., linear arrays, curved arrays, or phased arrays. The transducer array 114, for example, can include a two dimensional array (as shown) of transducer elements capable of scanning in both elevation and azimuth dimensions for 2D and/or 3D imaging. As is generally known, the axial direction is the direction normal to the face of the array (in the case of a curved array the axial directions fan out), the azimuthal direction is defined generally by the longitudinal dimension of the array, and the elevation direction is transverse to the azimuthal direction.

In some embodiments, the transducer array 114 may be coupled to a microbeamformer 116, which may be located in the ultrasound probe 112, and which may control the transmission and reception of signals by the transducer elements in the array 114. In some embodiments, the microbeamformer 116 may control the transmission and reception of signals by active elements in the array 114 (e.g., an active subset of elements of the array that define the active aperture at any given time).

In some embodiments, the microbeamformer 116 may be coupled, e.g., by a probe cable or wirelessly, to a transmit/receive (T/R) switch 118, which switches between transmission and reception and protects the main beamformer 122 from high energy transmit signals. In some embodiments, for example in portable ultrasound systems, the T/R switch 118 and other elements in the system can be included in the ultrasound probe 112 rather than in the ultrasound system base, which may house the image processing electronics. An ultrasound system base typically includes software and hardware components including circuitry for signal processing and image data generation as well as executable instructions for providing a user interface.

The transmission of ultrasonic signals from the transducer array 114 under control of the microbeamformer 116 is directed by the transmit controller 120, which may be coupled to the T/R switch 118 and a main beamformer 122. The transmit controller 120 may control the direction in which beams are steered. Beams may be steered straight ahead from (orthogonal to) the transducer array 114, or at different angles for a wider field of view. The transmit controller 120 may also be coupled to a user interface 124 and receive input from the user's operation of a user control. The user interface 124 may include one or more input devices such as a control panel 152, which may include one or more mechanical controls (e.g., buttons, encoders, etc.), touch sensitive controls (e.g., a trackpad, a touchscreen, or the like), and/or other known input devices.

In some embodiments, the partially beamformed signals produced by the microbeamformer 116 may be coupled to a main beamformer 122 where partially beamformed signals from individual patches of transducer elements may be combined into a fully beamformed signal. In some embodiments, microbeamformer 116 is omitted, and the transducer array 114 is under the control of the beamformer 122 and beamformer 122 performs all beamforming of signals. In embodiments with and without the microbeamformer 116, the beamformed signals of beamformer 122 are coupled to processing circuitry 150, which may include one or more processors (e.g., a signal processor 126, a B-mode processor 128, a Doppler processor 160, and one or more image generation and processing components 168) configured to produce an ultrasound image from the beamformed signals (i.e., beamformed RF data).

The signal processor 126 may be configured to process the received beamformed RF data in various ways, such as bandpass filtering, decimation, I and Q component separation, and harmonic signal separation. The signal processor 126 may also perform additional signal enhancement such as speckle reduction, signal compounding, and noise elimination. The processed signals (also referred to as I and Q components or IQ signals) may be coupled to additional downstream signal processing circuits for image generation. The IQ signals may be coupled to a number of signal paths within the system, each of which may be associated with a specific arrangement of signal processing components suitable for generating different types of image data (e.g., B-mode image data, Doppler image data). For example, the system may include a B-mode signal path 158 which couples the signals from the signal processor 126 to a B-mode processor 128 for producing B-mode image data.

The B-mode processor can employ amplitude detection for the imaging of structures in the body. The signals produced by the B-mode processor 128 may be coupled to a scan converter 130 and/or a multiplanar reformatter 132. The scan converter 130 may be configured to arrange the echo signals from the spatial relationship in which they were received to a desired image format. For instance, the scan converter 130 may arrange the echo signal into a two dimensional (2D) sector-shaped format, or a pyramidal or otherwise shaped three dimensional (3D) format. The multiplanar reformatter 132 can convert echoes which are received from points in a common plane in a volumetric region of the body into an ultrasonic image (e.g., a B-mode image) of that plane, for example as described in U.S. Pat. No. 6,443,896 (Detmer). The scan converter 130 and multiplanar reformatter 132 may be implemented as one or more processors in some embodiments.

A volume renderer 134 may generate an image (also referred to as a projection, render, or rendering) of the 3D dataset as viewed from a given reference point, e.g., as described in U.S. Pat. No. 6,530,885 (Entrekin et al.). The volume renderer 134 may be implemented as one or more processors in some embodiments. The volume renderer 134 may generate a render, such as a positive render or a negative render, by any known or future known technique such as surface rendering and maximum intensity rendering.

In some embodiments, the system may include a Doppler signal path 162 which couples the output from the signal processor 126 to a Doppler processor 160. The Doppler processor 160 may be configured to estimate the Doppler shift and generate Doppler image data. The Doppler image data may include color data which is then overlaid with B-mode (i.e. grayscale) image data for display. The Doppler processor 160 may be configured to filter out unwanted signals (i.e., noise or clutter associated with non-moving tissue), for example using a wall filter. The Doppler processor 160 may be further configured to estimate velocity and power in accordance with known techniques. For example, the Doppler processor may include a Doppler estimator such as an auto-correlator, in which velocity (Doppler frequency) estimation is based on the argument of the lag-one autocorrelation function and Doppler power estimation is based on the magnitude of the lag-zero autocorrelation function. Motion can also be estimated by known phase-domain (for example, parametric frequency estimators such as MUSIC, ESPRIT, etc.) or time-domain (for example, cross-correlation) signal processing techniques. Other estimators related to the temporal or spatial distributions of velocity such as estimators of acceleration or temporal and/or spatial velocity derivatives can be used instead of or in addition to velocity estimators. In some embodiments, the velocity and power estimates may undergo further threshold detection to further reduce noise, as well as segmentation and post-processing such as filling and smoothing. The velocity and power estimates may then be mapped to a desired range of display colors in accordance with a color map. The color data, also referred to as Doppler image data, may then be coupled to the scan converter 130, where the Doppler image data may be converted to the desired image format and overlaid on the B-mode image of the tissue structure to form a color Doppler or a power Doppler image.

According to examples of the present disclosure, output from the scan converter 130, such as B-mode images and Doppler images, referred to collectively as ultrasound images, may be provided to an completeness processor 170. The ultrasound images may be 2D and/or 3D. In some examples, the completeness processor 170 may be implemented by one or more processors and/or application specific integrated circuits. The completeness processor 170 may analyze the 2D and/or 3D images to detect/score task completeness, autorecord/automatically save video loops (e.g., a time series of images, cineloop), classify/score scan completeness, document scan completeness at the end of an exam, and/or a combination thereof.

In some examples, the completeness processor 170 may include any one or more machine learning, artificial intelligence algorithms, and/or multiple neural networks, collectively referred to as machine learning models (MLM) 172. The MLM 172 may include a deep neural network (DNN), a convolutional neural network (CNN), a recurrent neural network (RNN), an autoencoder neural network, or the like. The MLM 172 may be implemented in hardware (e.g., neurons are represented by physical components) and/or software (e.g., neurons and pathways implemented in a software application) components. The MLM 172 implemented according to the present disclosure may use a variety of topologies and algorithms for training the MLM 172 to produce the desired output. For example, a software-based neural network may be implemented using a processor (e.g., single or multi-core CPU, a single GPU or GPU cluster, or multiple processors arranged for parallel-processing) configured to execute instructions, which may be stored in computer readable medium, and which when executed cause the processor to perform a trained algorithm for identifying an organ, anatomical feature(s), and/or a view of an ultrasound image (e.g., an ultrasound image received from the scan converter 130). In some examples, the processor may perform a trained algorithm for identifying a zone and/or quality of an ultrasound image. In various embodiments, the MLM 172 may be implemented, at least in part, in a computer-readable medium including executable instructions executed by the completeness processor 170.

In some examples, MLM 172 may include You Only Look Once, Version 3 (YOLO V3) network. In some examples, YOLO V3 may be trained for organ and/or feature detection in images. The organ and/or feature detection may be used to determine whether a task has been completed (e.g., acquiring an image of the kidney-liver interface in the left upper quadrant during a FAST exam). In some examples, MLM 172 may include MobileNet network. In some examples, MobileNet may be trained for zone and/or image quality detection. In some examples, zone detection may be used to determine what zone (e.g., RUQ, LUQ, SP) of a subject is being imaged, and provide information on the tasks to be performed in said zone. In some examples, image quality detection may be used to determine whether an image including a recognized feature is of sufficient quality for diagnostic purposes.

In various examples, the MLM 172 may be trained using any of a variety of currently known or later developed learning techniques to obtain a neural network (e.g., a trained algorithm or hardware-based system of nodes) that is configured to analyze input data in the form of ultrasound images, measurements, and/or statistics. In some embodiments, the MLM 172 may be statically trained. That is, the MLM may be trained with a data set and deployed on the completeness processor 170. In some embodiments, the MLM 172 may be dynamically trained. In these embodiments, the MLM 172 may be trained with an initial data set and deployed on the completeness processor 170. However, the MLM 172 may continue to train and be modified based on ultrasound images acquired by the system 100 after deployment of the MLM 172 on the completeness processor 170.

In some embodiments, the completeness processor 170 may not include a MLM 172 and may instead implement other image processing techniques for feature recognition and/or quality detection such as image segmentation, histogram analysis, edge detection or other shape or object recognition techniques. In some embodiments, the completeness processor 170 may implement the MLM 172 in combination with other image processing methods. In some embodiments, the MLM 172 and/or other elements may be selected by a user via the user interface 124.

Outputs from the completeness processor 170, the scan converter 130, the multiplanar reformatter 132, and/or the volume renderer 134 may be coupled to an image processor 136 for further enhancement, buffering and temporary storage before being displayed on an image display 138. Although output from the scan converter 130 is shown as provided to the image processor 136 via the completeness processor 170, in some embodiments, the output of the scan converter 130 may be provided directly to the image processor 136.

A graphics processor 140 may generate graphic overlays for display with the images. According to examples of the present disclosure, based at least in part on the analysis of the images, the completeness processor 170 may provide display data for a list of tasks to be performed. The graphics processor 140 may provide the list of tasks as a text list next to or at least partially overlaying the image. In some examples, the completeness processor 170 may provide outputs to the graphics processor 140 to alter the displayed list of tasks as the completeness processor 170 determines tasks are completed. For example, the text associated with completed tasks may change color (e.g., from red to green), format (e.g., strikethrough), or no longer displayed as part of the list. In some examples, the completeness processor 170 may provide display information for additional feedback information to the graphics processor 140, such as completeness and/or quality scores.

Additional or alternative graphic overlays can contain, e.g., standard identifying information such as patient name, date and time of the image, imaging parameters, and the like. For these purposes the graphics processor may be configured to receive input from the user interface 124, such as a typed patient name or other annotations. The user interface 124 can also be coupled to the multiplanar reformatter 132 for selection and control of a display of multiple multiplanar reformatted (MPR) images.

The system 100 may include local memory 142. Local memory 142 may be implemented as any suitable non-transitory computer readable medium (e.g., flash drive, disk drive). Local memory 142 may store data generated by the system 100 including ultrasound images, executable instructions, imaging parameters, training data sets, or any other information necessary for the operation of the system 100. In some examples, the local memory 142 may store executable instructions in a non-transitory computer readable medium that may be executed by the completeness processor 170. In some examples, the local memory 142 may store ultrasound images and/or videos responsive to instructions from the completeness processor 170. In some examples, local memory 142 may store other outputs of the completeness processor 170, such as completeness scores.

As mentioned previously system 100 includes user interface 124. User interface 124 may include display 138 and control panel 152. The display 138 may include a display device implemented using a variety of known display technologies, such as LCD, LED, OLED, or plasma display technology. In some embodiments, display 138 may include multiple displays. The control panel 152 may be configured to receive user inputs (e.g., exam type, information calculated by and/or displayed from the completeness processor 170). The control panel 152 may include one or more hard controls (e.g., buttons, knobs, dials, encoders, mouse, trackball or others). In some embodiments, the control panel 152 may additionally or alternatively include soft controls (e.g., GUI control elements or simply, GUI controls) provided on a touch sensitive display. In some embodiments, display 138 may be a touch sensitive display that includes one or more soft controls of the control panel 152.

In some embodiments, various components shown in FIG. 1 may be combined. For instance, completeness processor 170, image processor 136 and graphics processor 140 may be implemented as a single processor. In some embodiments, various components shown in FIG. 1 may be implemented as separate components. For example, signal processor 126 may be implemented as separate signal processors for each imaging mode (e.g., B-mode, Doppler). In some embodiments, one or more of the various processors shown in FIG. 1 may be implemented by general purpose processors and/or microprocessors configured to perform the specified tasks. In some embodiments, one or more of the various processors may be implemented as application specific circuits. In some embodiments, one or more of the various processors (e.g., image processor 136) may be implemented with one or more graphical processing units (GPU).

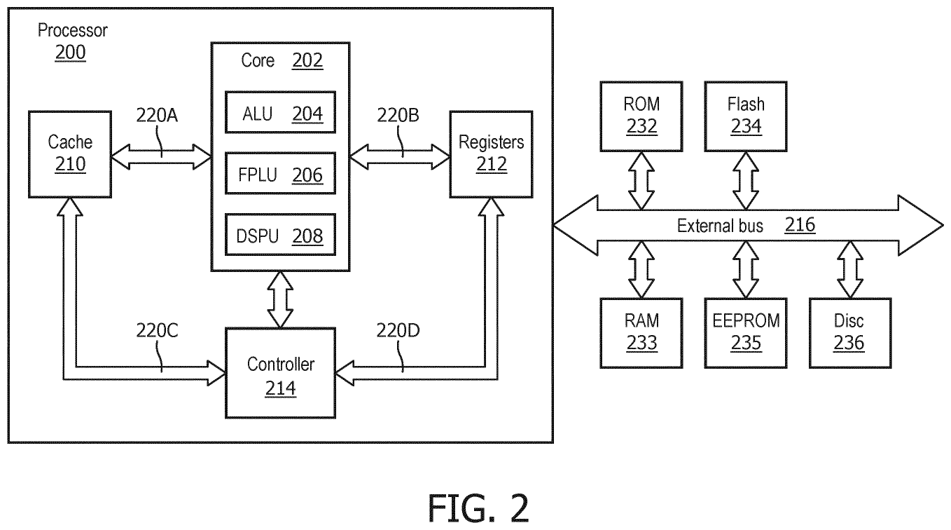

FIG. 2 is a block diagram illustrating an example processor 200 according to examples of the present disclosure. Processor 200 may be used to implement one or more processors and/or controllers described herein, for example, completeness processor 170, image processor 136 shown in FIG. 1 and/or any other processor or controller shown in FIG. 1. Processor 200 may be any suitable processor type including, but not limited to, a microprocessor, a microcontroller, a digital signal processor (DSP), a field programmable array (FPGA) where the FPGA has been programmed to form a processor, a graphical processing unit (GPU), an application specific circuit (ASIC) where the ASIC has been designed to form a processor, or a combination thereof.

The processor 200 may include one or more cores 202. The core 202 may include one or more arithmetic logic units (ALU) 204. In some embodiments, the core 202 may include a floating point logic unit (FPLU) 206 and/or a digital signal processing unit (DSPU) 208 in addition to or instead of the ALU 204.

The processor 200 may include one or more registers 212 communicatively coupled to the core 202. The registers 212 may be implemented using dedicated logic gate circuits (e.g., flip-flops) and/or any memory technology. In some embodiments the registers 212 may be implemented using static memory. The register may provide data, instructions and addresses to the core 202.

In some embodiments, processor 200 may include one or more levels of cache memory 210 communicatively coupled to the core 202. The cache memory 210 may provide computer-readable instructions to the core 202 for execution. The cache memory 210 may provide data for processing by the core 202. In some embodiments, the computer-readable instructions may have been provided to the cache memory 210 by a local memory, for example, local memory attached to the external bus 216. The cache memory 210 may be implemented with any suitable cache memory type, for example, metal-oxide semiconductor (MOS) memory such as static random access memory (SRAM), dynamic random access memory (DRAM), and/or any other suitable memory technology.

The processor 200 may include a controller 214, which may control input to the processor 200 from other processors and/or components included in a system (e.g., control panel 152 and scan converter 130 shown in FIG. 1) and/or outputs from the processor 200 to other processors and/or components included in the system (e.g., display 138 and volume renderer 134 shown in FIG. 1). Controller 214 may control the data paths in the ALU 204, FPLU 206 and/or DSPU 208. Controller 214 may be implemented as one or more state machines, data paths and/or dedicated control logic. The gates of controller 214 may be implemented as standalone gates, FPGA, ASIC or any other suitable technology.

The registers 212 and the cache 210 may communicate with controller 214 and core 202 via internal connections 220A, 220B, 220C and 220D. Internal connections may implemented as a bus, multiplexor, crossbar switch, and/or any other suitable connection technology.

Inputs and outputs for the processor 200 may be provided via a bus 216, which may include one or more conductive lines. The bus 216 may be communicatively coupled to one or more components of processor 200, for example the controller 214, cache 210, and/or register 212. The bus 216 may be coupled to one or more components of the system, such as display 138 and control panel 152 mentioned previously.

The bus 216 may be coupled to one or more external memories. The external memories may include Read Only Memory (ROM) 232. ROM 232 may be a masked ROM, Electronically Programmable Read Only Memory (EPROM) or any other suitable technology. The external memory may include Random Access Memory (RAM) 233. RAM 233 may be a static RAM, battery backed up static RAM, Dynamic RAM (DRAM) or any other suitable technology. The external memory may include Electrically Erasable Programmable Read Only Memory (EEPROM) 235. The external memory may include Flash memory 234. The external memory may include a magnetic storage device such as disc 236. In some embodiments, the external memories may be included in a system, such as ultrasound imaging system 100 shown in FIG. 1. For example local memory 142 may include one or more of ROM 232, RAM 233, EEPROM 235, flash 234, and/or disc 236.

In some examples, one or more processors, such as processor 200 may execute computer readable instructions encoded on one or more of the memories (e.g., memories 142, 232, 233, 235, 234, and/or 236). As noted, in some examples, processor 200 may be used to implement one or more processors of an ultrasound imaging system, such as ultrasound imaging system 100. In some examples, the memory encoded with the instructions may be included in the ultrasound imaging system, such as local memory 142. In some examples, the processor and/or memory may be in communication with one another and the ultrasound imaging system, but the processor and/or memory may not be included in the ultrasound imaging system. Execution of the instructions may cause the ultrasound imaging system to perform one or more functions. In some examples, a non-transitory computer readable medium may be encoded with instructions that when executed may cause an ultrasound imaging system to determine whether one or more anatomical features are included in an ultrasound image. Based on the anatomical features included in the ultrasound image, the ultrasound system may determine a status of a task, generate display data based on the status of the task, and cause a display, such as display 138, of the ultrasound imaging system to provide a visual indication of the status based on the display data. In some examples, some or all of the functions may be performed by one processor. In some examples, some or all of the functions may be performed, at least in part, by multiple processors. In some examples, other components of the ultrasound imaging system may perform functions responsive to control signals provided by the processor based on the instructions. For example, the display may display the visual indication based, at least in part, on data received from one or more processors (e.g., graphics processor 140, which may include one or more processors 200).

In some examples, the system 100 may be configured to implement one or more machine learning models, such as a neural network, included in the completeness processor 170. The MLM may be trained with imaging data such as image frames where one or more items of interest are labeled as present.

In some embodiments, a MLM training algorithm associated with the MLM can be presented with thousands or even millions of training data sets in order to train the MLM to determine a confidence level for each measurement acquired from a particular ultrasound image. In various embodiments, the number of ultrasound images used to train the MLM may range from about 1,000 to 200,000 or more. The number of images used to train the MLM may be increased to accommodate a greater variety of patient variation, e.g., weight, height, age, etc. The number of training images may differ for different organs or features thereof, and may depend on variability in the appearance of certain organs or features. For example, the organs of pediatric patients may have a greater range of variability than organs of adult patients. Training the network(s) to determine the pose of an image with respect to an organ model associated with an organ for which population-wide variability is high may necessitate a greater volume of training images.

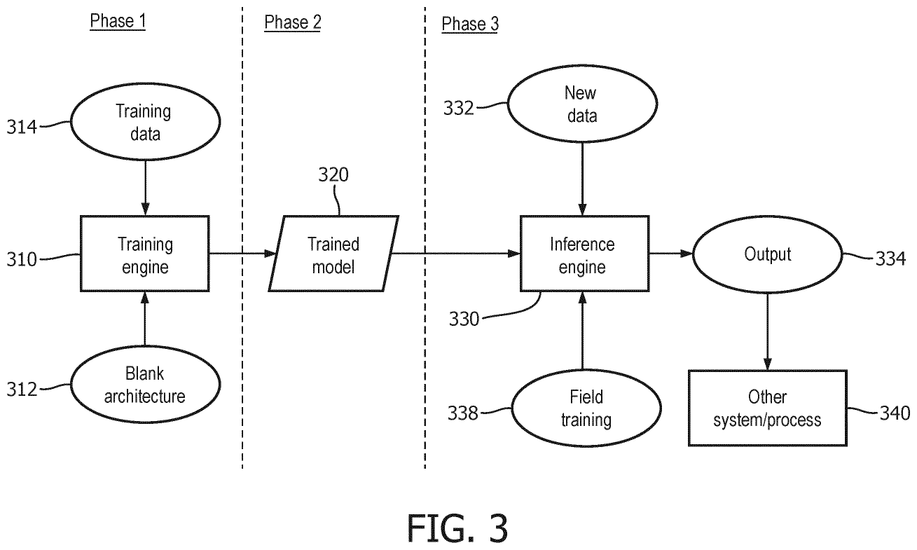

FIG. 3 shows a block diagram of a process for training and deployment of a machine learning model in accordance with examples of the present disclosure. The process shown in FIG. 3 may be used to train the MLM 172 included in the completeness processor 170. The left hand side of FIG. 3, phase 1, illustrates the training of a MLM. To train the MLM, training sets which include multiple instances of input arrays and output classifications may be presented to the training algorithm(s) of the MLM (e.g., AlexNet training algorithm, as described by Krizhevsky, A., Sutskever, I. and Hinton, G. E. “ImageNet Classification with Deep Convolutional Neural Networks,” NIPS 2012 or its descendants). Training may involve the selection of a starting (blank) architecture 312 and the preparation of training data 314. The starting architecture 312 may be a architecture (e.g., an architecture for a neural network with defined layers and arrangement of nodes but without any previously trained weights) or a partially trained network, such as the inception networks, which may then be further tailored for classification of ultrasound images. The starting architecture 312 (e.g., blank weights) and training data 314 are provided to a training engine 310 for training the MLM. Upon sufficient number of iterations (e.g., when the MLM performs consistently within an acceptable error), the model 320 is said to be trained and ready for deployment, which is illustrated in the middle of FIG. 3, phase 2. The right hand side of FIG. 3, or phase 3, the trained model 320 is applied (via inference engine 330) for analysis of new data 332, which is data that has not been presented to the model 320 during the initial training (in phase 1). For example, the new data 332 may include unknown images such as ultrasound images acquired during a scan of a patient (e.g., torso images acquired from a patient during a FAST exam). The trained model 320 implemented via engine 330 is used to classify the unknown images in accordance with the training of the model 320 to provide an output 334 (e.g., which anatomical features are included in the image, what zone the image was acquired from, quality of the image, or a combination thereof). The output 334 may then be used by the system for subsequent processes 340 (e.g., output of a MLM 172 may be used by the completeness processor 170 to provide a list of completed and outstanding exam tasks). In embodiments where the MLM 172 is trained, the inference engine 330 may be modified by field training data 338. Field training data 338 may be generated in a similar manner as described with reference to phase 1, but the new data 332 may be used as the training data. In other examples, additional training data may be used to generate field training data 338.

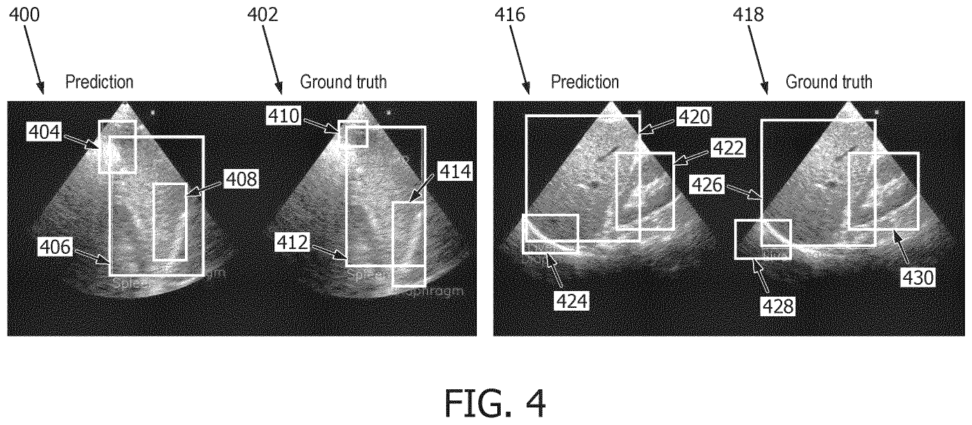

FIG. 4 shows example predictions made by a machine learning model compared to ground truth in accordance with examples of the present disclosure. Images 400 and 402 include ultrasound images of a spleen and diagraph acquired from a patient. The ultrasound images in images 400 and 402 are the same. However, image 400 indicates where anatomical features are predicted by a MLM, and image 402 indicates where the anatomical features are located as labeled by a trained observer (e.g., sonographer, radiologist), referred to as “ground truth.” Block 404 indicates where the MLM predicted the location of the spleen tip, and block 410 indicates where the spleen tip is “truly” located based on the labeling by the trained observer. Similarly, block 406 indicates the predicted location of the spleen and block 408 indicates the predicted location of the diaphragm. Block 412 indicates the “true” location of the spleen and block 414 indicates the “true” location of the diaphragm.

Images 416 and 418 include ultrasound images of a liver, diaphragm, and kidney. The ultrasound images in images 416 and 418 are the same. However, image 416 indicates predictions by a MLM, and image 418 indicates where the anatomical features are labeled by the trained observer. Block 420 indicates where the MLM predicted the location of the liver, and block 426 indicates the “true” location of the liver. Similarly, block 422 indicates the predicted location of the kidney and block 424 indicates the predicted location of the diaphragm. Block 430 indicates the “true” location of the kidney and block 428 indicates the “true” location of the diaphragm.

The predictions made in images 400 and 416 may be compared to the ground truth images 402 and 418 during training of the MLM, such as by the process described in FIG. 3. If the predictions made by the MLM in images 400 and 416 are within a desired margin of error of the ground truth images 402 and 418, the MLM may be determined to be trained and ready for validation and/or deployment.

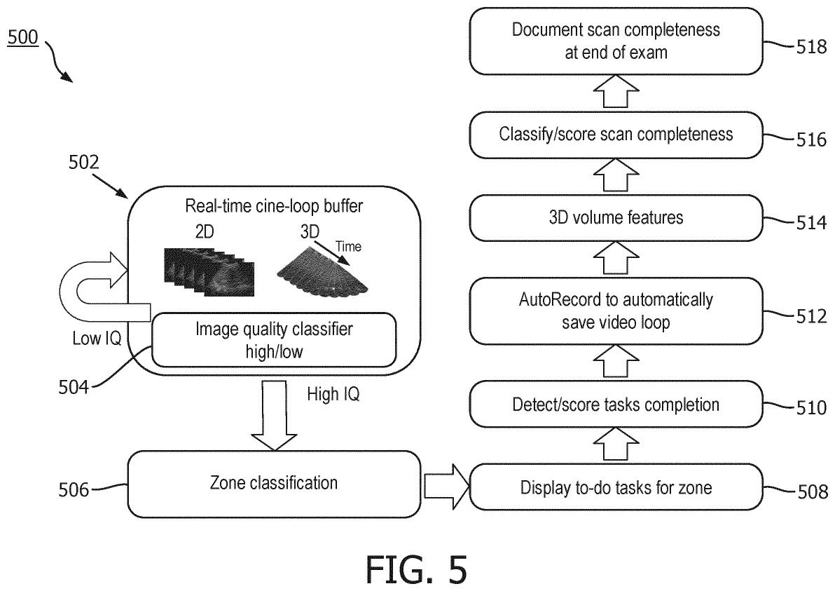

FIG. 5 is a flowchart providing an overview of exam completeness analysis in accordance with the examples of the present disclosure. In some examples, the tasks shown in flowchart 500 may be performed in whole or in part by one or more processor(s), such as completeness processor 170.

As indicated in block 502, the processor may receive real-time or near-real-time ultrasound data, such as a cineloop of 2D images or 3D images. The ultrasound images may be provided from a scan converter, such as scan converter 130. The scan converter may include a buffer that temporarily stores the images, and the images may be provided to the processor via the buffer in some examples.

The processor may determine whether the images are of high or low quality as indicated by block 504. For example, the signal-to-noise ratio, resolution, structural similarity index, or a combination thereof may be quality metrics that are calculated and used to assess image quality in some examples. In some examples, the calculated quality metric(s) may be compared to a threshold value to determine whether the images are of high or low quality. In some examples, the images may be determined to be of high or low quality based on whether the images include complete views of anatomy. For example, one or more MLM may detect anatomical features in the images, but may determine the anatomical features are not complete, or not all of the anatomical features required for analysis are present. For example, an MLM may detect a spleen is present in the image, but a tip of the spleen is not included. In another example, the MLM may detect a kidney, but may determine an interface with the liver is not present. If the images are determined to be low quality (e.g., the quality metrics are below a threshold value, incomplete anatomical features), the processor may wait for additional images to be acquired and perform the quality analysis again. In some examples, feedback may be provided to a user (e.g., text or graphics on a display, such as display 138), indicating new images are required.

When the images are determined to be of high quality (e.g., the quality metrics are equal to or above the threshold value, complete views of anatomy are included in the images), the processor may analyze the images (e.g., with MLM, such as MLM 172) to determine a zone being imaged as indicated by block 506. For example, whether the RUQ, LUQ, or SP zone is being imaged in a FAST exam. Other zones may be applicable to different exams (e.g., chambers of the heart may be different zones for a cardiac exam). In some examples, the processor may automatically detect what type of exam is being performed. In other examples, the type of exam may be indicated by a user input provided via user interface, such as user interface 124. In an example, a zone may be identified in block 506 or a type of exam associated with a particular view of a zone or feature may be identified in block 506. For example, while there may be a cardiac zone identified, a particular exam such as an exam using a 4-chamber view or a 2 chamber view may be identified within the same region. In an example, zone classification may occur based on feature or partial feature identification, detection and/or segmentation. Each of these particular identifications may be identified as part of block 506. Further, if one zone is identified and a user is at any point in the method 500, the user may independently decide to change the exam they are performing or the view they are identifying. For example, a user may decide to not complete a full exam before deciding to move to a completely different zone. In such case, a new zone may be identified or classified in block 506 and the method 500 would return to block 506 from another method block in method 500 in order to establish an updated exam process based on features identified in the image.

Based, at least in part, on the zone detected, the processor may cause a list of “to-do” tasks for the zone to be displayed as indicated by block 508. By task, it is meant a particular image, sequence, and/or measurement should be acquired (e.g., an image of hepato-renal interface). In some examples, the processor may implement one or more MLM for classification of the exam zones based on image features and provide the list of tasks to-do tasks for zone scan completion. In some examples, the list may be displayed as a prompt to the user or may be constantly displayed. In some examples, displaying the to-do task list may be dependent upon zone classification algorithm since list of tasks varies from zone to zone. In other examples, this feature may be offered independent of the zone classification algorithm when a user provides an input via the user interface to select a zone or particular exam to be performed. In some examples, zone information scan also be specified through a scan protocol sequence selected by the user or provided to the device via a remote user or system. As an example of a scan protocol sequence, medical standards for certain exams may dictate a specific order in which zones of a subject are scanned. The present techniques enable a particular exam protocol and its associated list of tasks to be provided for display and completeness assessment. Example lists of tasks to be completed for each zone for a FAST exam are provided in Table 1. In some examples, the task “Need Volume Fanning” can be shown as a to-do item when 2D image sequences are acquired. In some examples, volume fanning may be performed without any probe movements for 3D acquisitions as will be described in more detail with reference to block 514.

TABLE 1
Example To-Do Tasks for Different Zones

| RUQ | LUQ | SP |
|---|---|---|
| Kidney | Kidney | Bladder |
| Liver | Spleen | Uterus (Female) |
| Liver Tip | Spleen Tip | Transverse View |
| Diaphragm | Diaphragm | Sagittal view |
| Kidney-Liver Interface | Kidney-Liver Interface | Need Volume Fanning |
| Diaphragm-Liver Interface | Diaphragm-Spleen Interface | |
| Need Volume Fanning | Need Volume Fanning | |

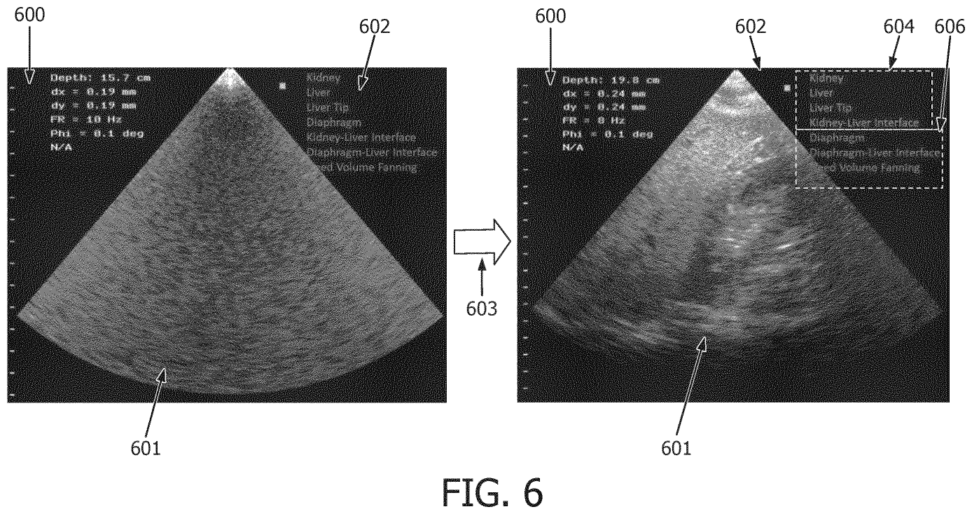

As indicated by block 510, the processor may detect and score task completion. The processor may use one or more MLM, such as MLM 172. In some examples, it may be assumed that there could be tasks which could not be completed as a detection task in a single image, for instance, detecting volume sweep or fanning, or imaging complete bladder volume. In some examples, the scoring/classification algorithm could be based upon rule-based approach (using output of anatomy detection algorithms) and/or a MLM trained specifically to classify or score tasks completion. Based on the analysis, the processor may updates progress of task completion on the display, as illustrated in FIG. 6. In an example, such as the case where a probe or transducer is moved to a new area, location, zone, or to initiate another exam other than a first exam identified, the method 500 may return to 506 for zone classification and a new list of tasks may be displayed in block 508 replacing the previously listed display tasks. Further, any partially completed exam may have its task completions stored in a memory such that a user may resume the exam or switch between zones and the previous status of tasks to display may be reloaded and displayed based on the detected zone being assessed. For example, if the kidney and liver were identified as imaging tasks completed in the RUQ of Table 1 and a user began scanning the LUQ zone to complete tasks then returning to the RUQ, the completed status of the Kidney and Liver tasks would be retained when the task list was again displayed. Further, the scan completeness and any image or loop storage undertaken as part of these tasks being completed could be stored despite the tasks not being completed in one continuous exam of the same zone. For example, a system may include a memory with which to store the status of a completed task in a set of tasks, where the completed task is and/or the set of tasks is stored as being associated with at least one of the zone or the anatomical feature identified.

Optionally, the processor may auto-record images acquired by an ultrasound imaging system (e.g., ultrasound imaging system 100) as indicated by box 512. While ultrasound systems typically include a buffer that retains the last several seconds of acquisitions (e.g., 5 seconds, 10 seconds), the images are overwritten or discarded if the user does not provide an input indicating the previously acquired images should be saved. In contrast, the process according to principles of the present disclosure, the processor may prospectively cause the next several seconds of acquisitions to be saved to memory (e.g., local memory 142) without requiring input from the user.

In some examples, the processor may utilize one or more MLM that performs anatomy detection/segmentation/classification and/or image quality classification/scoring to automatically detect key frames and record exam video loop (e.g., cineloop) without the user having to interact with the user interface. In some examples, the start of an exam may be detected by image quality (e.g., as discussed with reference to block 504) and images containing relevant anatomy (e.g., as discussed with reference to block 510), whereas end of exam can be triggered by scan completeness algorithm or manually by the user (e.g., as described with reference to blocks 516 and 518). This feature may reduce a number of manual interactions required during an exam. This may allow users to focus on analyzing images in real time (e.g., free-fluid exploration in a FAST exam) and/or reduce the risk of users forgetting to save a key image for review after the exam.

Block 514 may be performed by the processor when the ultrasound images are a 3D acquisition. The processor may utilize MLM that perform anatomy detection/segmentation/classification, partial anatomy detection/classification/scoring, and image quality classification/scoring to capture a complete zone without the user manipulating the probe (e.g., probe 112) and/or warning the user that a full zone cannot be scanned from the current position of the probe.

In some applications, block 514 may reduce or eliminate the need for manual volume fanning. In some types of exams, there may be key imaging location that can be used to acquire a complete volume scan to perform a complete exam (e.g., all zones or all tasks within the zone may be completed) without any probe movements. For example, a key imaging location in a FAST exam may be a probe location where the diaphragm, liver, and kidney are visible in a single image. The processor may analyze the 3D volume imagery and provide an output that indicates where a complete zone can be scanned from imaging point, warning user that a zone scan cannot be completed from the current probe location. If a complete exam is possible from the current probe position, the processor may cause the ultrasound imaging system to prompt the user to keep the probe stationary at this location and the ultrasound system automatically completes the scan.

Block 514 may be performed responsive to a user input or though live MLM that process 3D data in real-time. This MLM can be a rule-based or statistical analysis-based approach that makes use of outputs of anatomy and image quality classification algorithms or can be a standalone MLM that provides a binary flag or a confidence score that a complete scan can be acquired from this imaging point. In some examples, the images are not shown on display during the exam and the processor may cause the ultrasound imaging system to merely provide a report to the user about the contents of the 3D data and/or prompt the user to place the probe in another location.

The processor may use MLM to perform anatomy detection/segmentation/classification, partial anatomy detection/classification/scoring, image quality classification/scoring, and zone detection algorithms to classify/score zone scan completeness as indicated by block 516. This scoring/classification algorithm could be based upon rule-based approach (e.g., a number of tasks completed out of a total number of tasks assigned for scoring), statistical analysis, or MLM that can classify or score zone scan completeness based on image features computed by one or more MLM. The MLM-based features computation that enable zone classification and classification/scoring of zone scan completeness provide feedback to the user as the user is scanning, which may reduce or eliminate the need for input from an expert. The user interface features associated with block 514 may include classification into complete/incomplete and display a scan completeness score or scan meter that keep getting updated as the scan progresses. For example, text including “Complete” or “Incomplete” may be provided on a display. In another example, a status bar, area, or circle may gradually be filled in as the exam progresses. In a further example, text indicating a percentage completeness or score may be provided.

At block 518, the processor may provide exam completion related data at the end of exam. In some examples, the data may be saved as a complete/incomplete flag as part of the exam or scan completeness score can be saved as part of the exam, which may be saved, at least temporarily to a memory of the imaging system, such as local memory 142. However, the data, along with the exam data (e.g., images, annotations, etc.) may be transferred from the ultrasound imaging system to another computing system, such as a PACS system. Saving/documenting the scan completeness score/status may be used for filtering exams that need to be verified by an expert. For example, scan completeness scores may be compared to a threshold value. Exams having scan completeness scores equal to or above the threshold value may not be reviewed. In some applications, filtering which exams require expert review may reduce the experts' workloads. Additionally or alternatively, the completeness scores may be used to provide automated feedback to training/novice users.

In some examples, one or more of the various completeness scores (e.g., task and/or scan completeness scores) discussed herein may be calculated based one or more rules. For example, a scan completeness score may be based on a percentage of tasks completed (e.g., if 4 out of 5 required tasks are completed, the completeness score may be 80%). In some examples, one or more of the completeness scores may be based on confidence scores provided by the MLM. A confidence score is an output of the MLM that indicates a calculated accuracy of the prediction made by the MLM. For example, if an image acquired for a task has a 90% confidence score that the image includes the anatomical features required for the task (e.g., spleen tip), the completeness score for the task may be 90%. In some examples, a task may not be considered complete unless the confidence score is equal to or above a threshold value (e.g., 70%, 80%, 90%). In some examples, one or more of the completeness scores may be an average or weighted average of the confidence scores. For example, a completeness score for a zone may be based, at least in part, on an average of the confidence score associated with each task. Or, a task that requires multiple images to complete may have a completeness score that is an average of the confidence score for each image associated with the task.

FIG. 6 shows an example of a display providing a visual indication of tasks to be completed and completed in accordance with examples of the present disclosure. Display 600 may be included in display 138 in some examples, Display 600 provides an ultrasound image 601 acquired by an ultrasound imaging system (e.g., imaging system 100). Display 600 further provides a list 602 of to-do tasks. The list 602 may be based on a detected exam type and/or zone detected. As the exam progresses as indicated by arrow 603, the display 600 may alter the visual characteristics of list 602 to indicate which tasks have been completed. In the example shown in FIG. 6, the tasks that have been completed 604 in the list 602 are displayed in a different color (e.g., green) than the tasks that have not yet been completed 606 in the list 602. However, this is merely an example, and other techniques for indicating which tasks have been completed and which remain may be used in other examples. For example, completed tasks may “disappear” or may be crossed out.

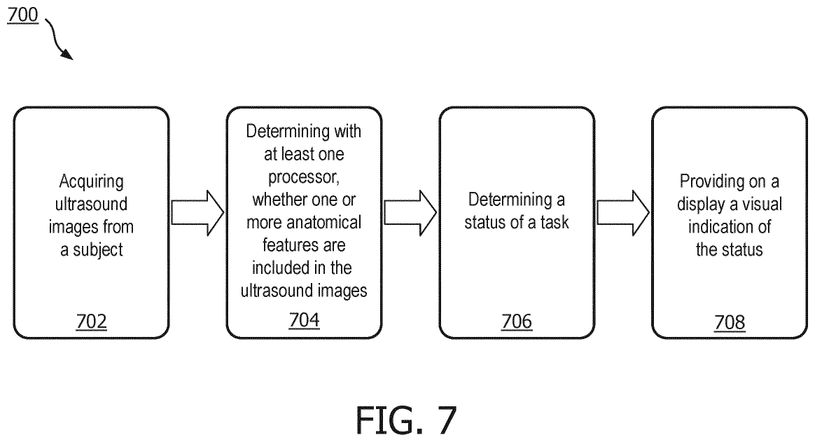

FIG. 7 is a flow chart of a method according to examples of the present disclosure. In some examples, the method 700 may be performed by an ultrasound imaging system, such as imaging system 100. In some examples, the method 700 may be performed in whole or in part by one or more processors, such as completeness processor 170 and/or graphics processor 140.

At block 702, “acquiring ultrasound images from a subject” may be performed. In some examples, the ultrasound images may be acquired with an ultrasound probe, such as ultrasound probe 112. In some examples, the ultrasound images may include one or more 2D images. In some examples, the ultrasound images may include one or more 3D images. In some examples, the ultrasound images may include a combination of 2D and 3D images.

At block 704, “determining with at least one processor, whether one or more anatomical features are included in the ultrasound images” may be performed. In some examples, the processor may include a completeness processor, such as completeness processor 170. In some examples, the processor may implement one or more machine learning models, such as MLM 172 to make the determination. In some examples, the processor may implement one or more image processing techniques (e.g., image segmentation) in addition to or instead of a machine learning model.

At block 706, “determining a status of a task” may be performed by the processor. In some examples, the determining may be based on the anatomical features included in the ultrasound images. For example, as discussed with reference to block 508 and 510 of FIG. 5.

At block 708 “providing on a display a visual indication of the status” may be performed. In some examples, the display may include display 138. In some examples, the processor may provide display data to the display based on the status of the task, and the display provides the visual indication of the status based on the display data. In some examples, the display also provides one or more of the ultrasound images. In some examples, the visual indication of the task and/or its status is provided at least partially overlaid on the image as shown in FIG. 6 and as discussed with reference to block 508 in FIG. 5.

In some examples, method 700 may further include determining a zone within the subject where the ultrasound images were acquired and providing a second visual indication of a set of tasks based on the zone. In some examples, the ultrasound imaging system may include a user interface, such as user interface 124, that is configured to receive an input from the user, and the input indicates an exam type, a zone of the subject, or a combination thereof. In some examples, a completed task of the set of tasks is displayed differently than an uncompleted task of the set of tasks. For example, as shown in FIG. 6, the completed task is a different color than the uncompleted task.

In some examples, method 700 may further include determining whether the ultrasound images are high quality or low quality. In some examples, it may be performed prior to determining whether the anatomical features are included. In some examples, the quality may be determined based on one or more quality factors (e.g., signal-to-noise). In some examples, determining the images are high quality or low quality may be based, at least in part, on whether the anatomical features are included. In some examples, a MLM may be used to determine the quality of the images. In some examples, the quality may be determined as described with reference to block 504 in FIG. 5.

In some examples, method 700 may include saving to a memory the ultrasound images, future acquired ultrasound images, or a combination thereof. For example, the ultrasound images may be saved to local memory 142. The images may be saved automatically as discussed with reference to block 512 in FIG. 5. In some examples, a MLM may be used to determine when to save the ultrasound images.

In some examples, the ultrasound images include a three-dimensional (3D) dataset. In some examples, the method 700 may further include determining, based on at least one ultrasound image (e.g., one or more images in the 3D data set, or a 2D image acquired prior to obtaining a 3D dataset), whether the task, a set of tasks, or a combination thereof can be completed by acquiring ultrasound images from a current location of the ultrasound probe. In some examples the determination may be made, at least in part, using a MLM. In some examples, method 700 may further include providing a prompt via a user interface to change a location of the ultrasound probe. For example, as described with reference to block 514 in FIG. 5.

In some examples, method 700 may further include computing a score indicating a degree of completeness of the task, a degree of completeness of an exam, or a combination thereof. For example, as described with reference to blocks 510 and 516 in FIG. 5. In some examples, the score is based, at least in part, on a confidence score provided by a MLM. In some examples, the score indicating a degree of completeness of the exam is based, at least in part, on a number of tasks completed out of a total number of tasks

While many of the examples provided herein refer to the FAST exam, the disclosure is not limited to FAST exams. For example, any ultrasound exam that has a set of standard images, videos, measurements, or a combination thereof, associated with the exam may utilize the features of the present disclosure.

In various embodiments where components, systems and/or methods are implemented using a programmable device, such as a computer-based system or programmable logic, it should be appreciated that the above-described systems and methods can be implemented using any of various known or later developed programming languages, such as “C”, “C++”, “C#”, “Java”, “Python”, and the like. Accordingly, various storage media, such as magnetic computer disks, optical disks, electronic memories and the like, can be prepared that can contain information that can direct a device, such as a computer, to implement the above-described systems and/or methods. Once an appropriate device has access to the information and programs contained on the storage media, the storage media can provide the information and programs to the device, thus enabling the device to perform functions of the systems and/or methods described herein. For example, if a computer disk containing appropriate materials, such as a source file, an object file, an executable file or the like, were provided to a computer, the computer could receive the information, appropriately configure itself and perform the functions of the various systems and methods outlined in the diagrams and flowcharts above to implement the various functions. That is, the computer could receive various portions of information from the disk relating to different elements of the above-described systems and/or methods, implement the individual systems and/or methods and coordinate the functions of the individual systems and/or methods described above.

In view of this disclosure it is noted that the various methods and devices described herein can be implemented in hardware, software and firmware. Further, the various methods and parameters are included by way of example only and not in any limiting sense. In view of this disclosure, those of ordinary skill in the art can implement the present teachings in determining their own techniques and needed equipment to affect these techniques, while remaining within the scope of the invention. The functionality of one or more of the processors described herein may be incorporated into a fewer number or a single processing unit (e.g., a CPU) and may be implemented using application specific integrated circuits (ASICs) or general purpose processing circuits which are programmed responsive to executable instruction to perform the functions described herein.

Although the present system may have been described with particular reference to an ultrasound imaging system, it is also envisioned that the present system can be extended to other medical imaging systems where one or more images are obtained in a systematic manner. Accordingly, the present system may be used to obtain and/or record image information related to, but not limited to renal, testicular, breast, ovarian, uterine, thyroid, hepatic, lung, musculoskeletal, splenic, cardiac, arterial and vascular systems, as well as other imaging applications related to ultrasound-guided interventions. Further, the present system may also include one or more programs which may be used with conventional imaging systems so that they may provide features and advantages of the present system. Certain additional advantages and features of this disclosure may be apparent to those skilled in the art upon studying the disclosure, or may be experienced by persons employing the novel system and method of the present disclosure. Another advantage of the present systems and method may be that conventional medical image systems can be easily upgraded to incorporate the features and advantages of the present systems, devices, and methods.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

What is claimed is:

1. An ultrasound imaging system comprising:
an ultrasound probe configured to acquire an ultrasound image from a subject;
a display configured to provide the ultrasound image; and
a processor configured to:
receive the ultrasound image;
determine whether one or more anatomical features are included in the ultrasound image;
based on the anatomical features included in the ultrasound image, determine a status of a task; and
provide display data to the display based on the status of the task,
wherein the display is further configured to provide a visual indication of the status based on the display data.

2. The ultrasound imaging system of claim 1, wherein the processor is further configured to:
determine a zone within the subject where the ultrasound image was acquired; and
provide second display data to the display based on a set of tasks associated with the zone,
wherein the display is further configured to provide a second visual indication of the set of tasks based on the second display data.

3. The ultrasound imaging system of claim 2, wherein a completed task of the set of tasks is displayed differently than an uncompleted task of the set of tasks.

4. The ultrasound imaging system of claim 1, wherein the processor implements a machine learning model configured to determine whether the anatomical features are included in the ultrasound image.

5. The ultrasound imaging system of claim 1, wherein the processor is further configured to determine whether the ultrasound image is high quality or low quality prior to determining whether the anatomical features are included.

6. The ultrasound imaging system of claim 1, further comprising a user interface configured to receive an input from the user, wherein the input indicates an exam type, a zone of the subject, or a combination thereof.

7. A non-transitory computer readable medium encoded with instructions that when executed, cause an ultrasound imaging system to:
determine whether one or more anatomical features are included in an ultrasound image;
based on the anatomical features included in the ultrasound image, determine a status of a task;
generate display data based on the status of the task; and
cause a display of the ultrasound imaging system to provide a visual indication of the status based on the display data.

8. A method comprising:
acquiring an ultrasound image from a subject with an ultrasound probe;
determining with at least one processor, whether one or more anatomical features are included in the ultrasound image;
based on the anatomical features included in the ultrasound image, determining a status of a task; and
providing on a display a visual indication of the status.

9. The method of claim 8, further comprising:
determining a zone within the subject where the ultrasound image was acquired; and
providing a second visual indication of a set of tasks based on the zone.

10. The method of claim 9, wherein a completed task of the set of tasks is displayed differently than an uncompleted task of the set of tasks.

11. The method of claim 10, wherein the completed task is a different color than the uncompleted task.

12. The method of claim 8, further comprising determining whether the ultrasound image is high quality or low quality prior to determining whether the anatomical features are included.

13. The method of claim 8, further comprising determining whether the ultrasound image is high quality or low quality based, at least in part, on whether the anatomical features are included.

14. The method of claim 8, further comprising saving to a memory the status of a completed task in a set of tasks stored as being associated with at least one of the zone or the anatomical feature identified.

15. The method of claim 8, wherein the ultrasound image comprises a three-dimensional (3D) dataset.

16. The method of claim 8, further comprising determining, based on at least one ultrasound image, whether the task, a set of tasks, or a combination thereof can be completed by acquiring a current ultrasound image from a current location of the ultrasound probe.

17. The method of claim 16, further comprising providing a prompt via a user interface to change a location of the ultrasound probe.

18. The method of claim 8, further comprising computing a score indicating a degree of completeness of the task, a degree of completeness of an exam, or a combination thereof.

19. The method of claim 18, wherein the score is based, at least in part, on a confidence score provided by a machine learning model.

20. The method of claim 18, wherein the score indicating a degree of completeness of the exam is based, at least in part, on a number of tasks completed out of a total number of tasks.
